(19) United States
(12) Patent Application Publication
(10) Pub. No.: US 2019/0298526 A1
(43) Pub. Date: Oct. 3, 2019

(54) APPARATUS AND METHODS FOR CREATION OF A PATIENT-SPECIFIC BODY PART OR MEDICAL INSERT

(71) Applicant: Mediprint, LLC, Chester, NY (US)

(72) Inventor: Brent Chanin, Goshen, NY (US)

(73) Assignee: Mediprint, LLC, Chester, NY (US)

(21) Appl. No.: 16/373,114

(22) Filed: Apr. 2, 2019

Related U.S. Application Data
(60) Provisional application No. 62/651,530, filed on Apr. 2, 2018.
(60) Provisional application No. 62/651,545, filed on Apr. 2, 2018.
(60) Provisional application No. 62/651,562, filed on Apr. 2, 2018.

(51) Int. Cl. A61F 2/30; B29C 64/393; B33Y 50/02; B33Y 80/00; G05B 19/4099

(52) U.S. Cl. CPC A61F 2/30942; B29C 64/393; A61F 2002/4435; B33Y 80/00; G05B 19/4099; B33Y 50/02

(57) ABSTRACT

Methods and apparatus for generating a patient-specific replacement body part. DICOM may be used to describe an anatomical portion of a patient and features may be differentiated that relate to a various biological systems. A user or a controller may designate a portion of features that relate to a biological system to be produced in a tangible model. A data file is generated that represents a surface geometry of a three-dimensional object representative of the portion of the features that relate to the biological system. Fabrication apparatus is operated to produce the object, which includes a tangible three-dimensional model of features that relate to the biological system.

### CROSS REFERENCES TO RELATED APPLICATIONS

This application claims priority to U.S. Provisional Patent Application Ser. No. 62/651,530, entitled “SYSTEM AND METHOD TO CREATE A PATIENT-SPECIFIC MEDICAL MODEL”, filed on Apr. 2, 2018 and to U.S. Provisional Patent Application Ser. No. 62/651,545 tilted “SYSTEM AND METHOD TO DESIGN A PATIENT-SPECIFIC REPLACEMENT BODY PART”, filed on Apr. 2, 2018 and to U.S. Provisional Patent Application Ser. No. 62/651,562 entitled “SYSTEM AND METHOD TO CREATE A PATIENT-SPECIFIC REPLACEMENT BODY PART”, each of which are relied upon and incorporated herein by this reference.

### FIELD OF THE DISCLOSURE

The present disclosure relates to a system and method to create a patient-specific replacement body part. The model is produced in vivo, non-invasively, and with sufficient accuracy and detail to assist in diagnosis and to create customized medical replacement body parts.

### BACKGROUND OF THE DISCLOSURE

Prosthetics have long been used to assist patients with damaged or diseased body parts. In general, prosthetics are made and sized according parameters meeting the norm for that body part. After manufacture, an appropriate size is selected for a particular patient.

More recently, augmented parts, such as orthotics or dental implants have been fashioned based upon molds made of a foot or mouth. However, more complex body parts, such as a spinal disk must be repaired using standard parts.

Anatomical models have many uses in the medical industry. Non-specific models of human physiology (i.e., models that are not limited to a specific person) are useful as learning tools in contexts such as anatomy class in medical school, medical instructions that affect a group of patients (e.g., for conditions such as pregnancy, insertion of an intra-uterine device, disorders such as enlarged hearts, etc.), cardio-pulmonary resuscitation (CPR) training, and so forth.

In contrast, a patient-specific medical model is a medical model of at least a portion of the anatomy (internal or external) of one specific patient, at one point in time. The patient-specific medical model may be useful to a medical practitioner to understand a patient's medical state, to plan a medical procedure for the patient, and/or to perform the medical procedure for the patient. A modality of the patient-specific medical model may refer to the technology used to obtain the patient-specific medical model.

One technology known to obtain a patient-specific medical model concludes computed tomography (CT) scan, which may also be referred to as computed axial tomography or computer aided tomography (CAT) scan. CT and CAT scans make use of computer-processed combinations of many X-ray measurements taken from different angles to produce cross-sectional (tomographic) images (virtual “slices”) of specific areas of a patient or other scanned object, allowing a medical professional to see inside the patient without cutting open the patient.

Another known technology for generating a patient-specific medical model is by magnetic resonance imaging (MRI). An MRI scanner uses strong magnetic fields, electric field gradients, and radio waves to generate images of the organs in the body. The images generally are of a planar slice through the patient's body. MRI does not involve X-rays and the use of ionizing radiation, which distinguishes it from CT or CAT scans. Compared with CT scans, MRI scans typically take longer and are louder, and may entail a patient being situated in a narrow, confining tube. In addition, patients with some medical implants or other non-removable metal inside the body may be unable to undergo an MRI examination safely.

Data from a CT scan or MRI scan is ordinarily stored in accordance with NEMA PS3/ISO 12052, Digital Imaging and Communications in Medicine (DICOM) Standard, National Electrical Manufacturers Association, Rosslyn, Va., USA (available free at http://medical.nema.org/). The DICOM standard covers both the formats to be used for storage of digital medical images and related digital data, and the protocols to be adopted to implement several communication services which are useful in the medical imaging workflow. DICOM facilitates cross-vendor interoperability among devices and information systems dealing with digital medical images.

Accurate and timely patient-specific medical modeling is sometimes extremely important to successful diagnosis or performance of a critical procedure such as a surgery, or treatment of delicate tissue such as the eye or nerve tissue. Surgeries may include cardiac surgery, neurosurgery, oncology surgery, ophthalmologic surgery, orthopedic surgery, plastic surgery, and so forth. A preferred physical resolution (e.g., slice thickness) in the background art of the patient-specific medical model may depend upon the type of surgery and the modality used to obtain the model data, and generally ranges from less than about 2.0 mm to less than about 0.7 mm.

A large drawback of known technologies is that systems and methods to convert patient-specific medical scans to actionable information is not well integrated, and often time-consuming to obtain. In addition, two dimensional scans are difficult to interpret and to visually ascertain important details. Other complexities may also be apparent to those with skill in the art.

### SUMMARY OF THE DISCLOSURE

Accordingly, the present disclosure provides for a method and apparatus to create a patient-specific body part that suitable for inclusion in the patient's physical body. The body part is typically of a size and shape and material that allows for the body part to replace or supplement a missing, damaged or diseased body part. The patient-specific body part may be manifested as a tangible three dimensional article that constitutes a size and shape congruent to an anatomical portion of a patient or a scaled size and shape with accurate reproduction of physical features present in a living organism modeled.

In some embodiments, the tangible three dimensional article and/or a model used to facilitate manufacture of the body part, may have different aspects color coded for ease of identification. In addition, different aspects of the three dimensional article may have include a texture and modulus that facilitates ease of identification or the aspect. For example, a blood vessel, such as an artery may be reproduced in a three dimensional model including a three dimensional (3D) printed article of a size and shape actually present in a patient. In addition, aspects of the article may be printed of different materials, for example, a modeled artery may be printed with a flexible material and a modeled bone may be printed with a rigid material.

Similarly, different aspects of a body part may be printed with material of specific characteristic in order to aid or alleviate a physical condition of a patient. For example, an artery may be 3D printed with a flexible material with a red hue and a vein may be 3D printed with a flexible material of a blue hue and bone may be printed in an ivory hue.

In general, according to the present invention, image scans are generated of an anatomical portion of a patient. The image scans may include homogenous or heterogeneous modalities, such as, for example one or more of: magnetic resonance imaging (MRI) and computed tomography (CT). The image scans may be stored as image data in standard DICOM format, or other format that enables data portability and interoperability.

In another aspect of the present invention, image scan data may be normalized by conversion to a common format such as DICOM or standard triangle language (“STL”) format. Data in STL format may be compatible with a fabrication apparatus such as a 3-D printer or further processed to be translated into a format compatible with a 3-D printer or other automated maker in order to directly create medical models or medical replacement parts from the imaging scans.

The details of one or more examples of the invention are set forth in the accompanying drawings and the description below. The accompanying drawings that are incorporated in and constitute a part of this specification illustrate several examples of the invention and, together with the description, serve to explain the principles of the invention: other features, objects, and advantages of the invention will be apparent from the description, drawings, and claims herein.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates a system to create a patient-specific body part, in accordance with some embodiments of the present invention;

FIGS. 2A-2B illustrate method steps to create a patient-specific model in accordance with some embodiments of the present invention.

FIG. 3 illustrates apparatus for designing a patient-specific body part, in accordance with an embodiment of the present invention; and

FIG. 4 illustrates a method to design a patient-specific body part, in accordance with an embodiment of the present invention.

FIG. 5 illustrates a method to create a patient-specific medical model, in accordance with an embodiment of the present invention.

### DETAILED DESCRIPTION

The present invention provides for apparatus and methods for manufacturing a tangible three dimensional (“3D”) article that constitutes a size and shape congruent to an anatomical portion of a patient; or an article that has a scaled size and shape with accurate reproduction of physical features present in a living organism modeled. A scaled article may be smaller or larger than the anatomical portion of the patient, but will accurately model features present in the anatomical portion of a patient and be suitable for inclusion as a body part of a patient. Manufacturing apparatus may include one or more of: additive manufacturing equipment, such as a 3D printer and a Computer numerical control (“CNC”) machine.

The tangible 3D article may be comprised of a single material, or a combination of materials. In some embodiments with multiple materials, aspects of the article may be created with a material that has qualities representative of a quality of the aspect of the anatomical portion of a patient. For example, an anatomical portion of a patient may include a spinal section of a patient. The article may include a hard plastic, ceramic, stainless steel, titanium or other rigid material to model aspects of the bone portions of a spine; and a nylon, resin or semi-rigid plastic to model a disc, and an artery and/or nerve may be manufactured from a flexible material, such as a thermoplastic polyurethane.

Embodiments may also include an anatomical portion of a patient that is manufactured to replace or supplement an actual body part. Continuing with our example of a spinal section of a patient, a disc shaped article may be 3D printed that is a same size and shape as a disc used to replace a damaged disc in the patient. The model may include the nerve and circulatory portions of the anatomy to scale and in position so that a surgeon may strategize a replacement procedure that will not collaterally damage the nerves and/or arteries. In addition, in some embodiments, the 3D printed disc may be used as the actual replacement disc. In such embodiments, the disc may be 3D printed to a size and shape specific to the patient.

An insert to supplement a body part may include, for example, a support for a spinal disc or other intersection of skeletal pieces.

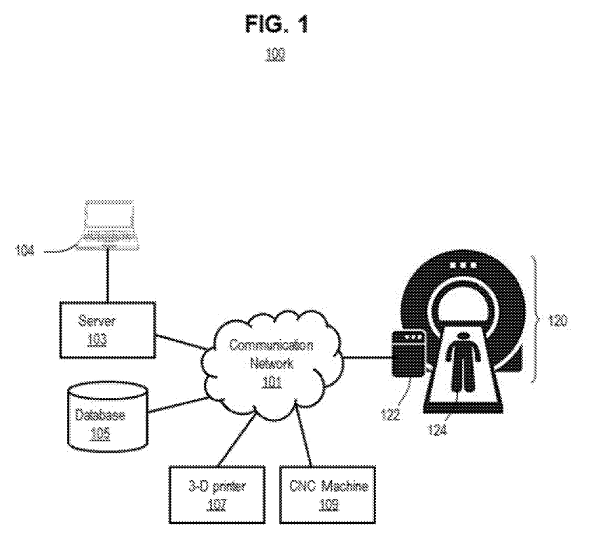

Referring now to FIG. 1 a system 100 to create a patient-specific body part is illustrated. System 100 includes a scanner capable of accurate representation of a portion of anatomy of a patient, such as an MRI machine 120. As illustrated the scanner is accommodating a patient 124 laying on a gurney and conducive to MRI based generation of image data. The MRI machine 120 may include a local control console 122, by which the local operation of MRI machine 120 may be controlled. For example, at local control console 122, an MRI operator (not shown) may control local operation such as movement of the patient gurney to move patient 124 into and out of MRI machine 120, starting and stopping other functions or operating modes of MRI machine 120, etc. Although FIG. 1 illustrates MRI machine 120 as the modality, other modalities such as a computed tomography scan (“CT scan”) machine are contemplated as being compatible with system 100.

MRI machine 120, or other scanner, communicatively interfaces with a communication network 101. Communication network 101 may include, e.g., at least a portion of an Ethernet network, the design of which is well known to persons of skill in the art of data networking. The communication interfaces to communication network 101, for MRI machine 120 and other elements of system 100, may be either wired (e.g., CAT-6 cable) or wireless (e.g., Wi-Fi in accordance with standards such as IEEE 802.11). Communication network 101 may represent various configurations such as more than one connected networks (e.g., multiple peer local area networks (LANs)), or networks of differing hierarchies (e.g., one or more LANs coupled to a wide area network (WAN)).

A controller 103 may be coupled to communication network 101. Controller 103 may be used by a system operator at terminal 104 to control operation of system 100. Terminal 104 may be coupled directly to controller 103 as illustrated, or terminal 104 may be coupled through communication network 101 to controller 103. Controlling the operation of system 100 may include, e.g., remote configuration of MRI machine 120 specific to the requirements for patient 124 (e.g., configuring the number of scans, field strength, duration of each scan, portion of the body to scan, scan plane, distance between adjacent scans, time between successive scans, data formatting requirements, etc.), and being able to study measurements made by MRI machine 120 and/or computations based upon the measurements. For example, an application program hosted on controller 103 may render a motion picture to illustrate time-based or position-based changes in successive scans.

System 100 further may include a database 105, in which image data generated via one or more MRI scans may be stored and subsequently retrieved. In some embodiments, image data may be stored in digital imaging and communications in medicine standard (“DICOM”) format or a DICOM compatible format.

In some embodiments, personally identifiable information (e.g., patient name) may be deleted or not saved with the scan data. In such embodiments, an identification number (e.g., patient ID number) may be saved with the scan data in order to associate the scan data with the correct patient. Database 105 may represent a single database, or a distributed database, such as a drive array or cloud storage.

System 100 further may include fabrication apparatus or subsystems to create physical models based upon scan data. For example, FIG. 1 illustrates a 3-D printer 107 and computer numeric control (CNC) machine 109 coupled to communication network 101. Controller 103 may include an application program that normalizes image data resulting from one or more scans. Some embodiments may include a model generated based upon reference to data generated by multiple scans. Moreover, in some embodiments, the multiple scans may be from disparate types of scanning apparatus, such as MRI, CT, X-Ray, sonogram etc. Controller 103 may convert disparate scan data formats in to a common format, such as for example, DICOM format. In addition, the controller 103 may convert the DICOM data into a file format compatible with a selected fabrication apparatus.

For example, a file format compatible with a fabrication apparatus may be based upon an unstructured triangulated surface by a unit normal and vertices or the triangles. The vertices may be ordered by a right-hand rule. The file format may be configured as three-dimensional Cartesian coordinates. The coordinates may be limited to positive values or include negative value. Associated file formats may include for example one or both of: binary and American Standard Code of Information Interchange (“ASCII”) file formats.

An ASCII file may include a facet normal n₁, n₂, n₃ (where each n is a floating point number) and respective vertexes: v₁, v₂, v₃ (each v value may also include a floating point number).

A binary file may describe multiple triangles via three 32-bit floating point numbers for the normal and three 32-bit floating point numbers for each vertex.

In some embodiments, file format compatible with a fabrication apparatus may include a standard triangle language (STL) format. The STL format file may be transmitted via logical communication to a fabrication apparatus, such as a 3-D printer 107 or CNC machine 109. The fabrication apparatus 107, 109 is operational to create a tangible 3-D object that is an anatomical model of an anatomical portion of a patient described by the DICOM data. The anatomical model may be to scale (e.g. 100% is life size), or at a designated scale selectable percentage of full size for examination purposes (e.g., 50% for a large body part, or 200% for a small body part). Accuracy of the 3-D object may be sufficient for the 3-D object to be used as a replacement body part. Replacement body parts may be ordinarily produced at 100% of full size; however, scaled sizes may also be generated. It is also possible to fabricate an object to be used to augment a patient's body that is not necessarily a replacement part. For example, an object may be fabricated that is complimentary to a size and shape of a body. Accordingly, an object may be attached to a body part that is not replacing a preexisting body part.

Although a 3-D printer and a CNC machine may be preferred modes of fabrication apparatus, other types of fabrication apparatus also are within the scope of FIG. 1. For example fabrication apparatus may include a stereo lithography machine, a voxel based lithography apparatus, a lathe or other manufacturing apparatus.

As an example of a use-case of system 100, a detailed patient-specific body part of the liver may be needed to facilitate diagnosis of disease and/or specifying a procedure to be performed that is specific to the anatomical attributes of a patient. For example, the patient may be under consideration for liver surgery, such as to remove cancerous tissue, or a liver transplant (as either a donor or recipient), or to remove damaged portions (e.g., by cirrhosis), or to repair damage such as a torn liver, and so forth.

To aid such procedures it is advantageous to generate a patient specific and time specific multi-layered model of the liver, e.g., a model of the patient-specific liver with blood vessels, a model of the patient-specific liver with nerves, and so forth. A time specific model may include, for example a time and date of scan data generated of the anatomical portion of the patient. The time designation is useful to model progression of one or both of patient recovery and advancement of a disease state.

These separate models may be helpful for different types of surgery, or for different phases of a lengthy surgery. The patient-specific model may be augmented with features to improve the usability or the stability of the model, such as by inclusion of physical support features. The physical support features may be rendered or produced in a way to include a feature not seen naturally, e.g., rendering the support features in a physical cross-sectional shape of polygons (such as squares, hexagons, octagons, etc.) in order to differentiate the physical support features from natural features of the patient-specific body part.

In some embodiments, the physical support features may include features that are usable to couple together different portions of the model. For example, the physical support features may include a detachable attachment feature, such as a hook-and-loop fastener (e.g., Velcro®). The feature may be produced directly by the fabrication apparatus (e.g., 3-D printer 107 prints the hook-and-loop fastener), or the fabrication apparatus may leave a surface or the like where a hook-and-loop fastener produced separately may be applied manually by a model maker.

In some embodiments, the patient-specific body part may be made from a highly flexible material, e.g., a material whose flexibility mimics the flexibility of (or conversely, the stiffness of) the actual body part that the patient-specific body part is intended to represent. For example, a patient-specific body part of blood vessels (either arteries or veins) or the lymph system may be fabricated in a double-wall or double-layer process. A first wall or layer (generically, “first layer”) may be fabricated in the shape of the vessel from a relatively stiffer material. A second wall or layer (generically, “second layer”) then may be fabricated adjacent to the first layer (usually, but not necessarily, outer to the first layer), and being fabricated in a way that is conformal to the first layer. The material used for the second layer may be relatively more flexible than the first layer.

In another aspect, a first layer may include a sacrificial layer such that after a second layer has been formed, the first layer optionally may be removed (such as by dissolving by a solvent such as acetone that does not significantly affect the second layer), thereby leaving the second layer as a highly flexible patient-specific body part of the blood vessels.

In some embodiments, the patient-specific body part made by the fabrication apparatus may have colors selected to provide emphasis to certain features. For example, a 3-D model of a patient-specific heart may be captured at a predetermined point in the heartbeat cycle, and may have clogged portions of various veins and arteries prominently colored if the purpose of the heart model is to plan an open-heart surgery, during which the clogged portions will be treated.

In other embodiments, a specific synthetic body part may be printed from the patient-specific medical model in order to use the synthetic body part as a replacement for a natural body part, e.g., one that is diseased, damaged, congenitally missing or disfigured, has to be replaced with a larger version as a juvenile patient grows, etc. The synthetic body part may be designed using a material that at least mimics observable or experiential characteristics of the natural body part, and may surpass the natural body part if appropriate. For example, synthetic body parts to replace cartilage-based body parts such as ears or the nose may be mimic the flexibility of the natural body parts. On the other hand, synthetic body parts to replace bony body parts such as elbows, kneecaps and spine vertebrae may be at least as hard as the natural body part. Synthetic body parts to replace a joint (e.g., knee or hip) may be made from materials having reduced friction during movement, compared to the natural body part.

In some embodiments, the imaging scans used to produce the patient-specific medical model may have been gathered with some disparateness in technical aspects of the imaging scans, e.g., gathered or saved with different data types, formats, intake protocols, varying levels of data validation and of anonymization of protected health information (PHI), and so forth. PHI is known as sensitive health information (e.g., a diagnosis of a psychiatric condition) that is personally identifiable with a specific person, and anonymization of PHI is known as the obscuring of data such that a risk of re-identifying the patient from the protected health information has been reduced below a configurable threshold. Disparities in the technical aspects may have arisen if, e.g., different MRI machines 120 were used (e.g., first at a trauma hospital and later at a reconstruction hospital), or if scans were taken in separate sessions in MRI machine 120, etc. Data validation scripts executing on controller 103 may be used to convert the disparate scan data into a common (i.e., shared) format so that the scan data is similar enough to be able to be shared and combined into an improved overall patient-specific medical model.

In some embodiments, aggregated scan and build data may be analyzed to produce an improved patient-specific body part. For example, artificial intelligence (“AI”) analysis techniques may be used to analyze the injury or disease state of a body part to be modeled or to be replaced, using an AI knowledge base stored in database 105. The AI knowledge base may include, e.g., average or typical scans from other patients, to be used for comparison sake. AI may be used to ascertain, for example, a disease state and/or structural abnormality.

This may be useful to produce an AI-guided model of therapeutic treatments, e.g., for a specific patient and based upon the patient's specific medical model, produce a prediction of success rate and possible complications from various modeled therapeutic treatments. For example, the AI-guided model may be able to model the alternative outcomes of surgery or no surgery (e.g., for orthopedic injuries), predicted treatment results over time (e.g., how soon after surgery a benefit will be observed, and how long that benefit will last after the surgery), and how the state of a disease or injury will advance over time (e.g., arthritis). These results from the AI-guided model may be compared to equivalent predictions based on professional judgment from a health care professional (e.g., a physician rendering an opinion within the area of their expertise) in order to improve over time the quality of the AI-guided model and/or the professional judgment.

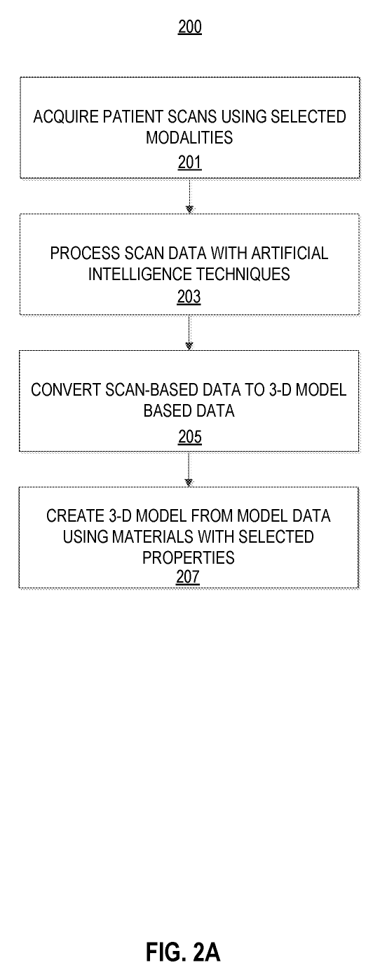

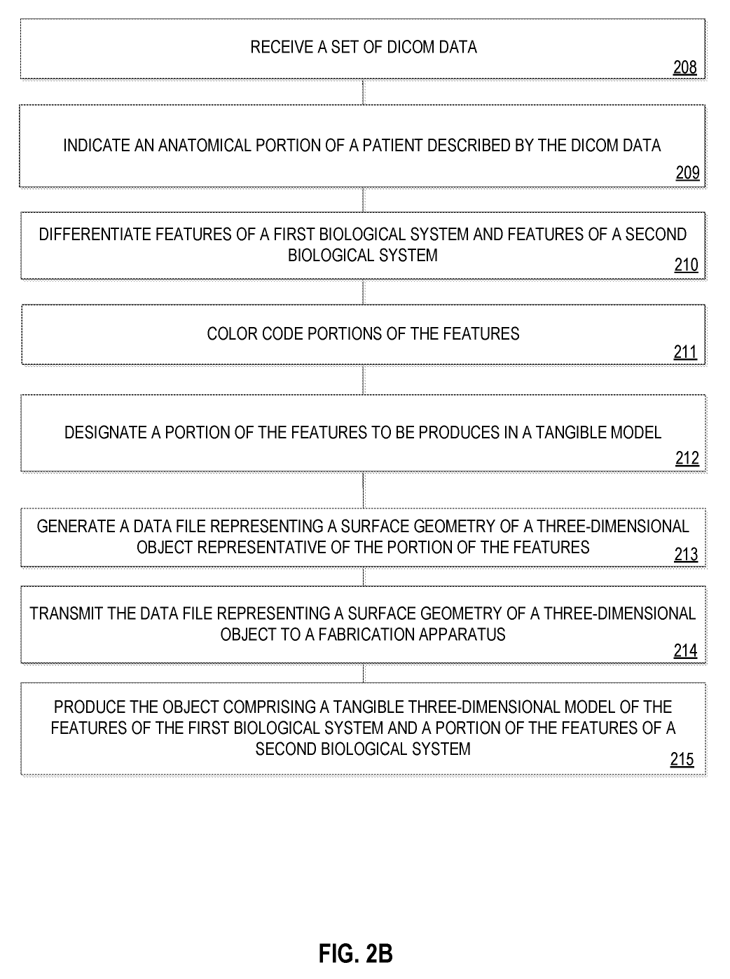

Referring now to FIG. 2A a process 200 is illustrated to create a patient-specific medical model and patient specific body part in accordance with some embodiments of the present invention. Process 200 begins at step 201, at which patient-specific scan data is acquired using a selected modality, e.g., by CT scan or by MRI scan. Process 200 continues to step 203, at which artificial intelligence techniques optionally may be applied to the scan data in order to improve the data quality, and to analyze the injury or disease state of body part to be modeled or to be replaced.

Next, process 200 may transition to step 205, at which the scan-based data of previous steps may be converted into a data format usable by a fabrication apparatus or subsystem, such as 3-D printer 107 and computer numeric control (CNC) machine 109, to create physical models based upon scan data. Next, process 200 may transition to step 207, at which a 3-D model may be created from the physical model data of step 205, using materials with selected properties.

Referring now to FIG. 2B, additional method steps are presented that may be executed in various implementations of the present invention. At step 208, a DICOM file may be received into a controller. The DICOM will be generated by an anatomical scanning machine, such as a MRI machine or a CAT scan machine, sonogram, x-ray machine and the like.

At step 209 a user may operate a user input device that is in logical communication with the controller to indicate an anatomical portion of interest that is included within the portion of the patient described by the DICOM data. The user input device may include, for example a trackball, a mouse, a pointer, and a keyboard of other apparatus that allows a human to interface with the controller. The user may additionally use the user input device to color code one or more features present within the DICOM data.

At step 210, differentiation of a first biological system and a second biological system may designated and include, by way of non-limiting example, designation of a bone structure, designation of nerves, designation of a blood vessel. In some embodiments, designation of a diseased feature may also be included within the differentiation of a first biological system and a second biological system. For example, a tumor, a cancer, diseased tissue, may be designated as a separate biological system.

In general, biological systems may include one or more of: a digestive system, a circulatory system, a muscular system, a skeletal system, a nervous system, an endocrine system, a respiratory system, an excretory system, and a reproductive system. In addition, systems may include one or more organs, glands, or muscles, including for example a liver, a pancreas, a heart, a blood vessel and an intestine. Other body parts may also be included in a system.

At step 211, with one of both of: operating a user input device; and via machine recognition; disparate colors may be allocated to disparate portions of the features. In some embodiments colors may allocated based upon inclusion in a biological system. Multiple biological systems may thus be visually differentiated based upon color contrast between different biological systems. For example, an artery may be colored red, a vein may be colored blue and a nerve tissue may be colored yellow while a bone is colored white and a cartilage is translucent. Other color schemes may also be assigned.

At step 212, a portion of the features may be designated as those portions included biological systems (such as a first biological system, a second biological system and a third biological system) that will be reproduced in a manufactured object, wherein the manufactured object becomes an anatomical model. Designation may be made for example via user interaction with a user interface, and/or via machine recognition based upon input of a desired biological system or system part, such as, for example, designation of a liver, or designation of a spine.

At step 213, a file may be generated that is representative of a surface geometry of a 3-D object representative of the designated portion of biological systems or other features that will be included in the manufactured object. The data file representing a surface geometry of a three-dimensional object may include a description of a triangulated surface via a unit normal and vertices of the triangles using a three-dimensional Cartesian coordinate system. A triangulated surface may be represented, for example as an ASCII file or a binary file. One format for the file may include an STL file.

A binary file may include a description of each triangle as twelve 32-bit floating-point numbers: three for the normal and then three for the X/Y/Z coordinate of each. A 2-byte (“short”) unsigned integer may also be included that is an attribute byte count.

At step 214, a file representing a surface geometry of a three-dimensional object to be produced is transmitted to a fabrication apparatus. Transmission may be accomplished, for example, via a digital network, such as an Internet Protocol digital network.

At 215 the three-dimensional object is produced. The object includes a tangible three-dimensional model of the features of the biological systems. The object may additionally include aspects of the objects in the colors specified in step 211 above. Other color variations may also be included. The object may also include support structures and sacrificial layers or portions of the manufactured object. Sacrificial portions may be removed via process suitable for that material included in the sacrificial portion, such as submersion in a liquid that will dissolve the sacrificial layer, but leave any non-sacrificial layers intact.

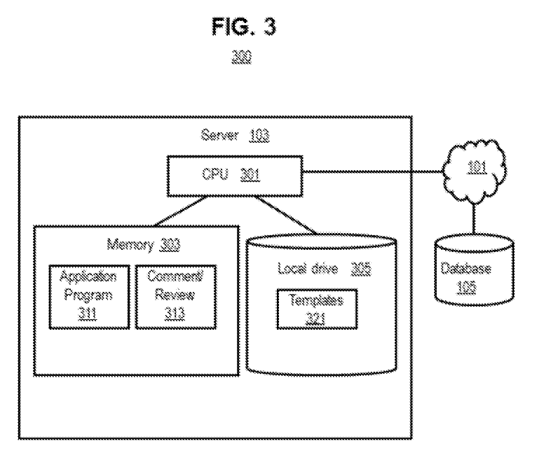

Referring now to FIG. 3 portions of system 100 are illustrated at a lower level of abstraction. In some embodiments, memory 303 of server 103 includes an application program 311 in the form of executable instructions that, when executed by central processing unit (CPU) 301, may perform the conversion of data as gathered from MRI machine 120 into models and instructions that can be used by a fabrication apparatus (e.g., 3-D printer 107 and/or computer numeric control (CNC) machine 109, or the like) in order to create a patient-specific body part. In particular, application program 311 may be used to design a patient-specific customized therapeutic part that can later be fabricated on a fabrication apparatus as a three-dimensional (3D) physical model of patient-specific anatomy, for use as a customized therapeutic part, to provide therapy or other medical treatment. The customized therapeutic part also may include portions that are assembled from separate pieces that are not necessarily created on the fabrication apparatus, e.g., pieces that may provide an adhesive, a weave, or a physical trait or material composition that may be difficult to duplicate with fabrication apparatus of the present art, or pieces that may be widely and inexpensively available from generic stock materials (e.g., wire, thread, filament, etc.). The choice of creating a portion of the customized therapeutic part by use of the fabrication apparatus or by assembly from separate pieces may be pre-configured or be controlled by a system operator. The assembly itself may be performed by a skilled human technician.

Server 103 further may include a local drive 305, which in turn may store templates 321. Templates 321 may be used as a library of standard models for generic body parts, i.e., body parts that are not patient specific and not necessarily derived just from data obtained by MRI machine 120. Standard models of generic body parts may be used when a patient-specific model is not needed for all body parts in a model. For example, if a model is needed for repairing knee ligaments, a standard model may be used to represent the knee cap.

Memory 303 of server 103 further may include an application program 313 in the form of executable instructions that, when executed by central processing unit (CPU) 301, may allow for the review, comment and editing of a model for a patient-specific customized therapeutic part, prior to its fabrication by a fabrication apparatus.

Embodiments in accordance with the present disclosure allow for review and practice of a medical procedure (e.g., a surgery) by a doctor, prior to performing the medical procedure on a live patient. Having a patient-specific customized therapeutic part also may facilitate additional rounds or levels of medical review before a medical procedure is performed on a patient. For example, the patient-specific customized therapeutic part may be used to consult with a more knowledgeable or experienced specialist, or to use for a peer review of a proposed surgery, and so forth. The consultation or review may be performed locally or remotely. The patient-specific customized therapeutic part also may be used to explain a proposed medical procedure to a patient, in order to better explain the procedure to the patient and in order to obtain a more informed patient consent.

The medical procedure also may involve replacing a body part with the patient-specific customized therapeutic part. In some circumstances, when a patient-specific customized therapeutic part is intended to be used for multiple purposes (e.g., to explain a procedure, and to use as a replacement for the body part), separate models may be fabricated. For example, for the purpose of explaining a procedure, a patient-specific customized therapeutic part may emphasize or make more prominent (e.g., by use of a bold color) any unusual anatomy or anticipated problems. On the other hand, a patient-specific customized therapeutic part intended for insertion into a patient may be fabricated from more inert materials, or from a material that is relatively more biologically compatible with human or animal tissue.

In some embodiments, the patient-specific customized therapeutic part may include support features to support other portions of the part. Such support features may be helpful to increase the robustness of otherwise delicate or fragile features. In some embodiments, the support features may be constructed with at least a portion being external to the rest of the patient-specific customized therapeutic part, e.g., provided as wires, threads, filaments, adhesive surfaces, and so forth. In other embodiments, the support features may be constructed with at least a portion being internal to the patient-specific customized therapeutic part, e.g., by use of a relatively stiffer material than the natural counterpart. A stiffer material may be used in concealed locations, e.g., on at least a portion of a concealed surface such as the interior of a vessel, bronchial tube, or other tube-like structures. The concealed surface also may be provided as an interior layer, similar to a fascia, or an outer surface similar to a shell. Such support features may be more useful for patient-specific customized therapeutic parts intended to be used for demonstrative purposes rather than to be used in vivo in the patient.

In some embodiments, the material used for the support surface may be selected to provide a selectable amount of support. For example, the support surface may have varying degrees of rigidity, pliability, softness, flexibility, and so forth. The support surface also may derive at least a portion of its support characteristics from being formed in a rigid lattice-like shape, such as a triangular or hexagonal tessellation.

In some embodiments, the support features may be visibly coded (e.g., by use of a non-natural color) in order to distinguish the support features from the natural portions of the patient-specific customized therapeutic part.

Embodiments in accordance with the present disclosure may include a continuation of anatomical features, such as for example via support structures and the like. For example, the continuation may be user-designated, such as made electronically on an editable MRI scan or on a computer model generated based upon normalized data from multiple MRI scans. Examples of such continuations may include blood vessels, nerves or other anatomical features, or proposed artificial items (e.g., stent, pacemaker, cochlear implant, brain stimulus probe, etc.) as they would be included in and around anatomical features. Such features may be AI designated and color coded to distinguish the feature from the rest of the patient-specific customized therapeutic part. Such features also may have a contiguous material and/or surface quality. As with support features described above, the continuation may be fabricated with a selectable amount of tactile characteristics such as varying degrees of rigidity, pliability, softness, flexibility, and so forth. The continuation also may derive at least a portion of its tactile characteristics from being formed in a rigid lattice-like shape, such as a triangular or hexagonal tessellation.

Embodiments in accordance with the present disclosure also may facilitate user control of a three-dimensional model used by the fabrication apparatus. For example, embodiments may allow a user to select a portion of a scan or a model, for generation as a physical representation of the patient-specific customized therapeutic part, based upon normalized data from multiple MRI scans.

Embodiments may allow a user to print only selected portions of the patient-specific customized therapeutic part. For example, a user may be allowed to pan, rotate, zoom, etc. selected portions of the patient-specific customized therapeutic part in order to better illustrate a portion of greater interest. Some embodiments may fabricate outer layers of the patient-specific customized therapeutic part in a transparent or translucent material, in order to make at least partially visible an interior structure of interest. Some embodiments may fabricate portions of the patient-specific customized therapeutic part in special colors. For example, a cancerous organ may be rendered with the cancerous portion of the organ rendered in a different color to visually highlight it. In some embodiments, the patient-specific customized therapeutic part may omit section, portions, features or the like in order to better illustrate features of interest. For example, a patient-specific customized therapeutic part fabricated as a bone in order to illustrate a bone marrow transplant procedure may be fabricated in cutaway form, such that the marrow interior of the bone is visible.

Embodiments may design a patient-specific portion of the body, not limited to a single organ. For example, embodiments may include all features in a given area of the body, or multiple specific organs (e.g., related organs like the liver, gall bladder, and pancreas). bones with attached ligaments, etc. In some embodiments, features of less interest may be omitted in order to concentrate attention on the features of greater interest.

In some embodiments, aggregated scan and build data may be analyzed to produce an improved patient-specific body part. For example, artificial intelligence (“AI”) analysis techniques may be used to analyze the injury or disease state of a body part to be modeled or to be replaced, using an AI knowledge base stored in database 105. The AI knowledge base may include, e.g., average or typical scans from other patients, to be used for comparison sake. This may be useful to produce an AI-guided model of therapeutic treatments, e.g., for a specific patient and based upon the patient's specific medical model, produce a prediction of success rate and possible complications from various modeled therapeutic treatments. For example, the AI-guided model may be able to model the alternative outcomes of surgery or no surgery (e.g., for orthopedic injuries), predicted treatment results over time (e.g., how soon after surgery a benefit will be observed, and how long that benefit will last after the surgery), and how the state of a disease or injury will advance over time (e.g., arthritis). These results from the AI-guided model may be compared to equivalent predictions based on professional judgment from a health care professional (e.g., a physician rendering an opinion within the area of their expertise) in order to improve over time the quality of the AI-guided model and/or the professional judgment.

Embodiments in accordance with the present disclosure facilitate the selection of materials used in 3D models. For example, material characteristics or material features of the materials may be matched to corresponding characteristics or features of the replacement body part. Material characteristics and material features may include hardness, softness, firmness, flexibility, tackiness, slipperiness, smoothness, roughness, friction or resistance to wear (especially for joints), ductility, brittleness, density, color, porosity, tempered, annealed, tensile strength, molecular or microstructure characteristics (e.g., grain, fibers, nanolattice), thermal resistance, hydrophobic or hydrophilic, radiopacity, and so forth. Certain material characteristics may be avoided when possible. For example, usage of a material attracted to a magnet (e.g., a steel rod) may be avoided. Such materials may be problematic in the presence of magnetic fields (e.g., at a metal detector, in an MRI machine, etc.).

Specific anatomical parts, organs, features, etc., may be characterized in terms of body part characteristics, and the body part characteristics may be associated with the closest available material characteristics or material features. Alternatively, the body part characteristics may be associated with at least a threshold level of the corresponding material characteristic or material feature. Alternatively, the body part characteristic further may be associated with a material having preferably a greater amount of the material characteristic or material feature.

For example, bone may be characterized by an amount of stiffness, or an amount of stiffness per unit cross-sectional area. A major weight-bearing bone may such as a tibia, a femur, or a vertebra in the lower back may have an amount of stiffness that is greater than the stiffness of a non-weight-bearing bone such as a collarbone, a rib, a metacarpal or phalanges bone, etc. A replacement bone part may be made from a material having at least as much stiffness as the corresponding natural bone, and preferably a greater amount of stiffness.

In another example, a replacement joint may have an amount of friction that is less than the friction of a corresponding natural joint, and/or a resistance to wear that is greater than the resistance the wear of the corresponding natural joint. For such a body part, a greater amount of a desirable characteristic (e.g., resistance to wear) is better, and a lesser amount of an undesirable characteristic (e.g., friction) is better.

For some body parts (e.g., an external body part having at least a portion as an external skin surface), it may be preferable to mimic a characteristic of a natural body part as closely as possible by appropriate selection of material rather than to exceed the characteristic of the natural body part. For example, a prosthetic hand (or ear) preferably should have a texture, softness, firmness, flexibility, tackiness, etc., that matches a natural hand (or ear). Consequently, a silicone or similar material may be useful for an external body part. As a counterexample, a prosthetic hand that is too stiff may be perceived as being claw-like.

For some body parts, it may be preferable to select a material color that matches the color of the original body part. For example, it is more important to match a color of a visible body part such as an ear or prosthetic hand, compared to the color for an internal body part such as a bone, an organ, a stent, etc. In some embodiments, a non-natural color may be used for an internal body part in order to easily identify it as an artificial part during any future surgeries. In some embodiments, several different colors may be used for external body parts because skin tone is not one consistent color but a mixture of textures and colors. Skin around the knuckles, for example, usually has more red pigment, as do palms. The palm of the hand is also commonly lighter in color than the back of the hand and the arm.

In some embodiments, a body part model to be used for illustration or teaching purposes, but not for usage as an operational body part, may be fabricated from a material having a different material characteristic or material feature than the corresponding natural body part. For example, a model of an internal organ (e.g., liver, kidney, etc.) may be fabricated with a non-natural color in order to highlight its position within the torso.

In some embodiments, an application program such as application program 311 may be used to help create the patient-specific body part. For example, application program 311 may have access to one or more of a library of material characteristics for various fabrication materials, and a library of normal characteristics for various natural body parts. The one or more libraries may be stored in a persistent memory such as database 105 or local drive 305. Application program 311 may offer to a user a selection of materials to use when fabricating the patient-specific body part. A best match or suggested match may be offered, based upon a comparison of at least one material characteristic with normal or typical characteristics for the body part. In some embodiments, the selection may be modified by specific abnormal characteristics of a patient-specific body part. For example, if a patient-specific model of a liver scarred with cirrhosis or a skin surface scarred with psoriasis is desired, the material selection for fabrication may be modified based upon the condition the model should include. The selection of fabrication materials may be made by a user using familiar user interface GUI controls such as a drop-down selection list. The selection of fabrication materials also may be influenced by the physical size of the model being produced. For example, if a large portion of the body is being produced, or a relatively smaller portion (e.g., an organ) is being produced at a larger-than-life size, then additional physical support features may be needed. Preferably, any additional physical support features will be provided in a way that is not readily visible to a casual observer.

In some embodiments, fabrication of a model may depend upon a user-controlled quality setting. For example, a high quality setting may produce a model having superior realism for as many body part characteristics as possible. Such a model may be slower to produce, or may use more expensive materials. In other circumstances, when a faster or less expensive model is sufficient, a model may be produced with a lower quality setting.

In some embodiments, suitable materials for a replacement body part and/or a body part model may include one or more of silicone, plastic, metal, ceramic, and so forth. All materials should include biocompatibility with human tissue. In some embodiments, a replacement body part such as artificial skin may be fabricated at least in part with a culture of human cells or compatible animal cells (e.g., pig cells) grown on a substrate mesh or membrane.

In some embodiments, a body part model may be fabricated from a luminescent material in order to facilitate illuminating at least a portion of the body part model. In some embodiments, luminescent portions of a body part model may be designed in coordination with light emitting diodes (LEDs) or other lighting sources. For example, LEDs may be either embedded within the body part model as internal illumination, or as external illumination of the body part model, or a combination of both lighting locations. In some embodiments, the body part model may be fabricated to be compatible with a handheld light source, e.g., by having sufficient clearance for the movement of a handheld light source.

In some embodiments, when a replacement body part is being fabricated for use by a patient, a choice and selection of materials may depend upon whether the replacement body part is intended for internal usage (e.g., as orthopedic repair) or for external usage (e.g., as orthopedic support). In some embodiments, a replacement body part and a body part model may be fabricated either sequentially or simultaneously (subject to fabricator apparatus availability), from the same patient-specific body part.

In some embodiments, when a body part model or a replacement body part requires a fastener (such as a screw for, e.g., a dental implant) to secure together pieces or to secure the part to a bone or similar rigid part, application program 311 may design the placement and size of the fastener, and/or a place for the fastener to attach (such as a threaded screw hole). The fasteners themselves may be conventionally-supplied medical grade fasteners.

In some embodiments, application program 311 may facilitate the selection of FDA-approved parts based upon the patient-specific medical model, provide a list of any additional required parts in order to verify that the necessary parts and components are on hand and available before a surgery. Doing so helps allow for peer review prior to surgery; helps obtain more informed patient consent prior to surgery, and may be able to save time during the surgery itself.

Embodiments in accordance with the present disclosure are not limited to the creation of a patient-specific medical model only for use as a replacement body part or for illustration during training or instructions. Embodiments are also useful to create a patient-specific medical model for use as, e.g., prototypes, final use parts, and emergency usage parts (possibly on a temporary basis) in trauma or triage situations. Fabrication apparatus and its usage will comply with applicable law and regulation, and also preferably adhere to best practices, with respect to: equipment operator identification, qualifications, and training; qualifications and certifications such as ISO 9000; maintenance requirements, logs, and schedules; and material chain of custody such as material sourcing, material quality assurance (QA), and material storage.

Manufacturing QA, including monitoring and observation, may be performed with technological apparatus where feasible, such as video monitoring and recording, sensors to detect flaws or assure compliance of individual parts with a design, and provide real-time observation capability to a system operator. The fabrication apparatus will undergo routine maintenance such as scheduled maintenance, cleanings, minor repairs as needed, disposal of replaced parts, and maintaining records thereof including at least some of operator or technician identification, title, dates, etc. Maintenance tools, equipment, and methods may include: usage of clay tools and brushes; pressure washing; usage of pumps, tanks or buckets; ultrasonic cleaning; sterilization (e.g., by heat, or chemicals such as alcohol or bleach); and so forth. Cleaning and maintenance procedures will maintain sterility, adhere to biohazard standards when and where applicable, and dispose of waste in a manner commensurate with its risk. If necessary, cleaning and maintenance procedures may be performed by guided or imaging tools, such as a remote controlled cleaning robot. Where feasible and without creating a biohazard, waste materials may be recycled in accordance with prevailing technology, standards, and initiatives.

In some embodiments, a body part model may be fabricated with an aesthetically pleasing appearance (e.g., a glossy finish) or physical texture (e.g., smooth), as long as the aesthetic artifacts (e.g., surface reflectivity or surface micro-texture) are not important to the demonstrative purpose of the body part model. For example, if a body part model of a liver is used only to illustrate the location of the liver in the abdominal cavity and its placement with respect to other internal organs, then the surface reflectivity and surface micro-texture of the liver model is not important. Standard model-making tools and methods may be used to provide the aesthetically pleasing appearance, such as sanding (e.g., grits of 100-5000), polishing and polish compounds, polishing cloths and sponges, etc. Power tools (e.g., oscillatory or rotary saws, sanders, etc.) may be used. Priming (e.g., heat or chemical) and painting (e.g., spray, brush, roller, airbrush, etc.) may be used.

The fabrication apparatus, its usage, its interface to other portions of system 100, and its scheduling may be automated to the extent feasible (e.g., if justified from a cost/benefit analysis) compared to alternative methods such as a manual method.

In some embodiments, post-process validation may include a manual measurement to verify that parts are being fabricated according to design. For example, validation may include parts scanning (e.g., a laser-based 3D measurement), and a computational comparison of the measurement to the design. Machine learning techniques may be used, e.g., if a systematic bias is found in the 3D measurements. Token verification may also be utilized.

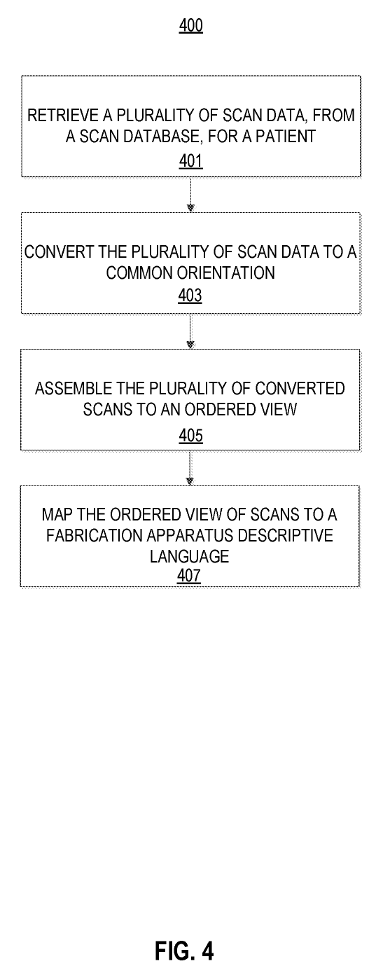

FIG. 4 illustrates a process 400 that may be used to design a patient-specific replacement body part. Process 400 may begin at step 401, at which retrieving a plurality of scan data, from scan database 105, for a patient. Next, process 400 proceeds to step 403 at which server 103 may convert the plurality of scan data to a common orientation. For example, the scan data may be converted to a predetermined angular view, resolution, size, and so forth. Next, process 400 proceeds to step 405 at which server 103 may assemble the plurality of converted scans to an ordered view. For example, the converted scans may be reordered to a sequential set of slices from top to bottom of a knee joint. Next, process 400 proceeds to step 407 at which server 103 may map the ordered view of scans to a fabrication apparatus descriptive language.

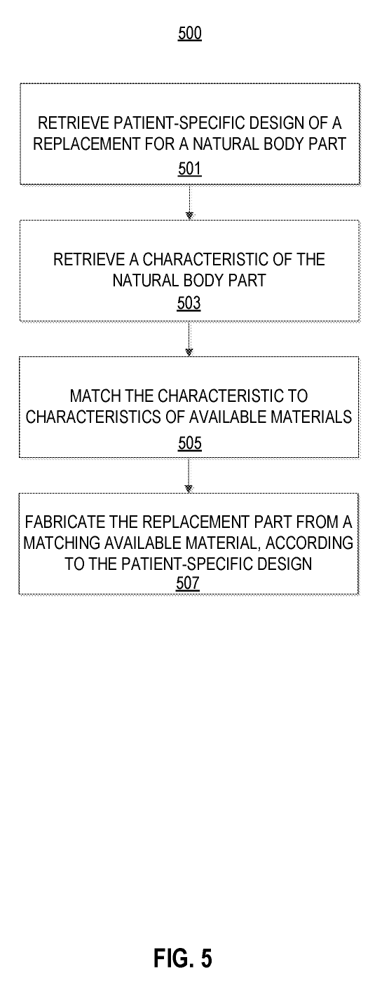

FIG. 5 illustrates a process 500 that may be used to design a patient-specific replacement body part. Process 500 may begin at step 501, at which a patient-specific design of a replacement for a natural body part may be retrieved. The design may be retrieved from a non-volatile memory such as database 105, and the design may be created by an application such as application program 311, using data obtained from system 100.

Next, control of process 500 progresses to step 503, at which a characteristic of the natural body part may be retrieved. For example, a texture of a patient's natural skin may be retrieved. Additional characteristics of the natural body part may be retrieved, such as color or softness. Specific characteristics that are retrieved may be dependent upon, and appropriate to, the natural body part. As a counterexample, hardness would be unlikely to be appropriate for natural skin.

Next, control of process 500 progresses to step 505, at which the retrieved characteristic(s) of the natural body part is compared to, and matched to, the corresponding characteristics of materials that might be available to fabricate a patient-specific replacement body part. The required closeness of a matching material selected from the available materials may be controlled by a designer using the system, e.g., by setting a tolerance level for the maximum deviation between the characteristic of the natural body part and the characteristic of the available material.

Next, control of process 500 progresses to step 507, at which the patient-specific replacement body part may be fabricated from the selected matching material, according to the patient-specific design.

Particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed invention.

What is claimed is:

1) Method for producing patient-specific body part, comprising:
a) receiving a set of DICOM data into a controller comprising a processor and a memory, the memory storing instructions to be executed by the processor;
b) operating a user input device in logical communication with the controller to indicate an anatomical portion of a patient described by the DICOM data;
c) in the DICOM data, designate features that relate to a patient body part;
d) designating a portion of the features that relate to the patient body part to be produced as a tangible object;
e) generating a data file representing a surface geometry of a three-dimensional object representative of the patient body part to be produced as a tangible body part;
f) via a digital communications network, transmitting the data file representing a surface geometry of a three-dimensional object to a fabrication apparatus; and
g) with reference to the data file representing a surface geometry of the three-dimensional object, operating the operating fabrication apparatus to produce the three dimensional object, said three dimensional object comprising a tangible three-dimensional embodiment of the features that relate to the patient body part.

2) The method of claim 1 additionally comprising the step of receiving into a user interface a user input specifying a color to be associated with the portion of the features of the three-dimensional object representative of the patient body part to be produced as a tangible body part.

3) The method of claim 2, wherein the data file representing a surface geometry of a three-dimensional object comprises a description of a triangulated surface via a unit normal and vertices of triangles using a three-dimensional Cartesian coordinate system.

4) The method of claim 3, wherein the data file comprises an ASCII file.

5) The method of claim 3 wherein the data file includes facet normals n₁, n₂, n₃, wherein each n is a floating point number, and respective vertexes: v₁, v₂, v₃, wherein each v value includes a floating point number.

6) The method of claim 3, wherein the data file comprises binary file.

7) The method of claim 6, wherein the triangles are represented via three 32-bit floating point numbers for a normal and three 32-bit floating point numbers for each vertex.

8) The method of claim 2, wherein the patient body part to be produced comprises a replacement portion of bone.

9) The method of claim 8, wherein the patient body part to be produced as a tangible body part comprises a replacement portion of a patient's circulatory system

10) The method of claim 9, wherein the patient body part to be produced as a tangible body part comprises a support for a spinal disc.

11) An apparatus for producing patient-specific body part, comprising:
a controller comprising a processor and a memory, and a communication device for receiving a set of DICOM data into the controller and the memory storing instructions to be executed by the processor; the software operative with the processor to:
a) receive a logical communication into the controller the logical communication comprising an indication of an anatomical portion of a patient described by the DICOM data;
b) in the DICOM data, designate features that relate to an anatomical portion of a patient to be produced as a body part;
c) generate a data file representing a surface geometry of a three-dimensional object representative of the designated features that relate to an anatomical portion of a patient to be produced as a body part;
d) via a digital communications network, transmit the data file representing a surface geometry of a three-dimensional object to a fabrication apparatus, said data file including sufficient information to operate the fabrication apparatus to produce a body part comprising the three-dimensional object representative of the designated features.

12) The apparatus of claim 11 wherein the software is additionally operative with the processor to receive into a user interface a user input specifying a color to be associated with the portion of the features of the three-dimensional object representative of the patient body part to be produced as a tangible body part.

13) The apparatus of claim 12, wherein the data file representing a surface geometry of a three-dimensional object comprises a description of a triangulated surface via a unit normal and vertices of triangles using a three-dimensional Cartesian coordinate system.

14) The apparatus of claim 13, wherein the data file comprises an ASCII file.

15) The apparatus of claim 13 wherein the data file includes facet normals n₁, n₂, n₃, wherein each n is a floating point number, and respective vertexes: v₁, v₂, v₃, wherein each v value includes a floating point number.

16) The apparatus of claim 13, wherein the data file comprises binary file.

17) The apparatus of claim 16, wherein the triangles are represented via three 32-bit floating point numbers for a normal and three 32-bit floating point numbers for each vertex.

18) The apparatus of claim 12, wherein the body part to be produced as a tangible object comprises a replacement portion of bone.

19) The apparatus of claim 18, wherein the patient body part to be produced comprises a replacement portion of a patient's circulatory system.

20) The apparatus of claim 19, wherein the patient body part to be produced comprises a support for a spinal disc.
